(19) United States
(12) Patent Application Publication
(10) Pub. No.: US 2023/0012491 A1
(43) Pub. Date: Jan. 19, 2023

(54) CORRECTION OF IMAGES AND DEPTH INFORMATION FOR DETECTION WITH MATRIX

(71) Applicant: SMITHS DETECTION FRANCE S.A.S., Vitry Sur Seine (FR)

(72) Inventors: Serge MAITREJEAN, Vitry Sur Seine (FR); Sébastien GRABEUIL, Vitry Sur Seine (FR); Cindy BERGERAT, Vitry Sur Seine (FR)

(21) Appl. No.: 17/784,770

(22) PCT Filed: Dec. 11, 2020
(86) PCT No.: PCT/GB2020/053189

§ 371 Date: Jun. 13, 2022

(30) Foreign Application Priority Data
Dec. 13, 2019 (GB) 1918415.9

(51) Int. Cl. A61B 6/00; G06T 5/00; G06T 5/50

(52) U.S. Cl. CPC A61B 6/5241; G06T 5/002; G06T 5/50; G06T 2207/10116

(57) ABSTRACT

In some examples, there is described a method for processing inspection data associated with cargo irradiated by a plurality of successive pulses of X-rays. The method may involve obtaining the inspection data, the inspection data being generated as a result of scanning the cargo using a matrix including a plurality of at least two rows of detectors, and a source of the plurality of successive pulses. In some examples radiation corresponding to the plurality of successive pulses irradiating the cargo is arranged in a first order on the plurality of rows of detectors of the matrix and one or more successive reconstruction zones for the inspection data and corresponding to different orders are determined. Intermediate images of the cargo and an average image are generated. On the generated average image, pixels may be selected and neighbourhoods of the pixels having fewer artefacts may be extracted.

### CROSS-REFERENCE TO RELATED APPLICATIONS

This application is a national stage application of PCT/GB2020/053189 filed on Dec. 11, 2020, which claims priority to GB Application No. 1918415.9 filed on Dec. 13, 2019, the disclosures of which are hereby incorporated by reference herein in their entirety as part of the present application.

### BACKGROUND

The disclosure relates but is not limited to a method for inspecting cargo with X-rays. The disclosure also relates but is not limited to a corresponding inspection system and a corresponding computer product or a computer program.

Inspecting cargo with X-rays may be performed by scanning the cargo by mutually displacing the cargo and a scanner including a detector, with a mutual scanning displacement, and detecting, with the scanner, radiation generated by a plurality of successive X-ray pulses irradiating the cargo during the mutual scanning displacement. A matrix of detectors including a plurality of at least two rows of detectors may be used for inspection.

As illustrated in FIG. 1A and FIG. 1B, a projection of a point A or B of a cargo 10 on a matrix 1 depends on a distance z of the point A or B from the matrix 1. As illustrated in FIG. 1A, points A and B are projected on a row 12 of the matrix 1. As illustrated in FIG. 1B, when the point A located at a distance z corresponding to a middle plane is moving a distance d during the scan, the point A is projected on a row 13 of the matrix 1. However when the point B located at a distance z closer to the matrix 1 is moving the same distance d during the scan, the point B is projected on the row 12 of the matrix 1. It should be understood that the two points A and B which were situated on the same line of projection for one pulse on FIG. 1A (and therefore represented by a single point in a corresponding image) are dissociated for the X-ray pulse of FIG. 1B. The dependency of the projections on depth introduces distortions in a corresponding final image of the cargo.

The distortions in the final image are also due to the fact that, for generating the final image of the cargo, the arrangement of data is performed with respect to a reconstruction plane. Selecting the reconstruction plane corresponds to considering that the whole cargo is located in the reconstruction plane. Therefore, zones of the cargo which are located in the reconstruction plane appear without distortion on the final image. However zones of the cargo which are located in other planes than the reconstruction plane will appear with distortions in the final image because of the dissociation of points of the cargo explained above. Oversampling of data increases the above distortions, because some parts of the cargo are irradiated several times and will appear several times in the final image.

FIG. 2 shows the matrix 1 moving and the cargo 10 not moving during the mutual scanning displacement. In other words FIG. 2 corresponds to a mobile mode, but the same explanations would also apply to a pass-through mode. In order to avoid the oversampling mentioned above, as illustrated in FIG. 2, the matrix 1 may be used such that a frequency of the X-ray pulses is adapted to a speed of the matrix 1 during the mutual scanning displacement x. The frequency of the X-ray pulses may be calculated such that a displacement of the matrix 1 between two pulses corresponds approximately to a width of the matrix 1 of detectors. However, adjusting the frequency of the X-ray pulses to the speed of the matrix 1 as just explained has as a consequence that zones 15 of the cargo 10 are not imaged.

### BRIEF DESCRIPTION

In one embodiment, a method for processing inspection data associated with cargo irradiated by a plurality M of successive pulses of X-rays is provided. The method includes obtaining the inspection data, the inspection data being generated as a result of scanning the cargo using a matrix including a plurality N of at least two rows of detectors, and a source of the plurality M of successive pulses, the matrix being located at a distance D from the source, the scanning including mutually displacing the cargo and the matrix with a mutual scanning displacement, and detecting, with the matrix, radiation corresponding to the plurality M of successive X-ray pulses irradiating the cargo, during the mutual scanning displacement, wherein, in the inspection data, radiation corresponding to the plurality M of successive pulses irradiating the cargo and detected by the plurality N of at least two rows of detectors is arranged in a first order corresponding to a level of the matrix, in a direction corresponding to the mutual scanning displacement. The method further includes determining one or more successive reconstruction zones for the inspection data, wherein each reconstruction zone corresponds to a range of distances from the source in which radiation corresponding to the plurality M of successive pulses irradiating the cargo is arranged in an order which is different from the first order and different from that of another reconstruction zone of the one or more successive reconstruction zones, selecting one or more reconstruction planes, based on the determined one or more reconstruction zones, for each selected reconstruction plane, generating an intermediate image of the cargo, generating an average image by averaging all of the generated intermediate images, on the generated average image, selecting one or more pixels having a gradient, in a direction corresponding to the mutual scanning displacement, greater in absolute value than a predetermined threshold, and for each one of the selected pixels: extracting a neighbourhood of the selected pixel from each generated intermediate image, and determining, in the extracted neighbourhoods, the neighbourhood minimizing a criterion compared with the other extracted neighbourhoods.

In another embodiment, a system is provided. The system includes a processor, and a memory storing instructions which, when executed by the processor, enable the processor to perform a method for processing inspection data associated with cargo irradiated by a plurality M of successive pulses of X-rays. The method includes obtaining the inspection data, the inspection data being generated as a result of scanning the cargo using a matrix including a plurality N of at least two rows of detectors, and a source of the plurality M of successive pulses, the matrix being located at a distance D from the source, the scanning including mutually displacing the cargo and the matrix with a mutual scanning displacement, and detecting, with the matrix, radiation corresponding to the plurality M of successive X-ray pulses irradiating the cargo, during the mutual scanning displacement, wherein, in the inspection data, radiation corresponding to the plurality M of successive pulses irradiating the cargo and detected by the plurality N of at least two rows of detectors is arranged in a first order corresponding to a level of the matrix, in a direction corresponding to the mutual scanning displacement. The method further includes determining one or more successive reconstruction zones for the inspection data, wherein each reconstruction zone corresponds to a range of distances from the source in which radiation corresponding to the plurality M of successive pulses irradiating the cargo is arranged in an order which is different from the first order and different from that of another reconstruction zone of the one or more successive reconstruction zones, selecting one or more reconstruction planes, based on the determined one or more reconstruction zones, for each selected reconstruction plane, generating an intermediate image of the cargo, generating an average image by averaging all of the generated intermediate images, on the generated average image, selecting one or more pixels having a gradient, in a direction corresponding to the mutual scanning displacement, greater in absolute value than a predetermined threshold, and for each one of the selected pixels extracting a neighbourhood of the selected pixel from each generated intermediate image, and determining, in the extracted neighbourhoods, the neighbourhood minimizing a criterion compared with the other extracted neighbourhoods.

Aspects and embodiments of the disclosure are set out in the appended claims. These and other aspects and embodiments of the disclosure are also described herein.

### BRIEF DESCRIPTION OF DRAWINGS

Embodiments of the present disclosure will now be described, by way of example, with reference to the accompanying drawings, in which:

FIG. 1A, already discussed, schematically illustrates projections of points of a cargo irradiated by a first X-ray pulse, the projections being on a matrix of detectors;

FIG. 1B, already discussed, schematically illustrates projections of the points of FIG. 1A irradiated by a second X-ray pulse;

FIG. 2, already discussed, schematically illustrates a cargo irradiated by a first X-ray pulse and a second X-ray pulse, the frequency of the pulses being selected to avoid oversampling;

FIG. 3 shows a flow chart illustrating an example method according to the disclosure;

FIG. 4 schematically illustrates a cargo irradiated by a first X-ray pulse and a second X-ray pulse, the frequency of the pulses being selected to avoid parts of the cargo not being imaged;

FIGS. 5A and 5B illustrate examples of artefacts depending on a choice for a plane of reconstruction for intermediate images;

FIG. 6 illustrates an example neighbourhood according to the disclosure;

FIG. 7 shows another flow chart illustrating an example method according to the disclosure;

FIG. 8 schematically illustrates an example final image with a superimposed depth image; and

FIG. 9 schematically illustrates an example system configured to implement a method according to any aspect of the present disclosure.

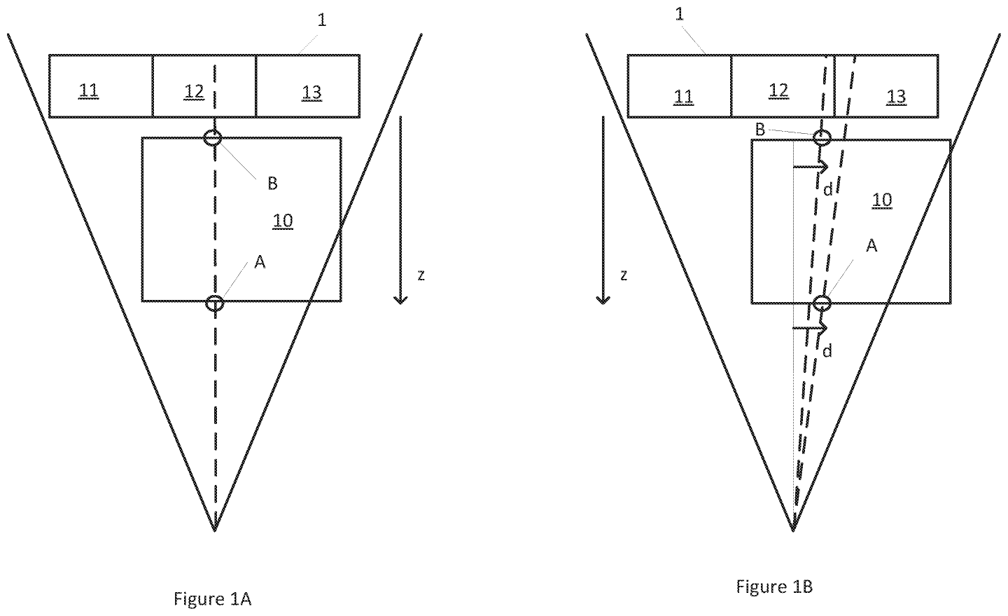

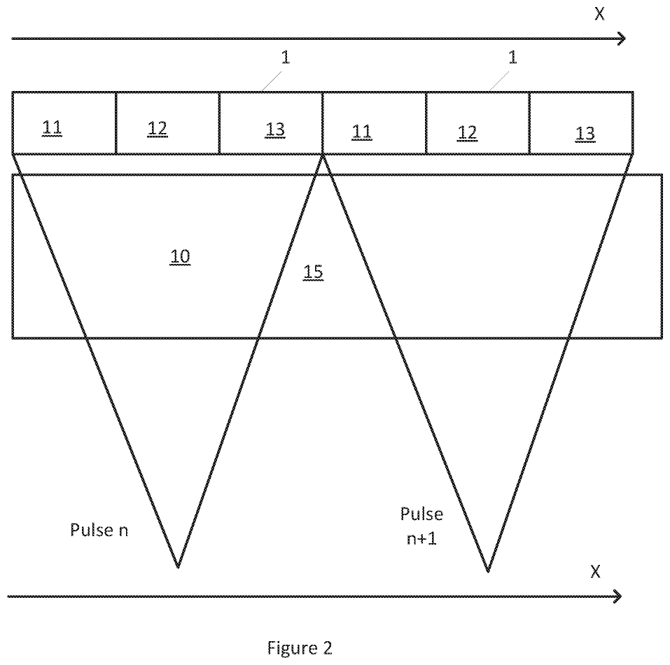

In the figures, similar elements bear identical numerical references.

### DETAILED DESCRIPTION

### Overview

Embodiments of the disclosure provide a method for processing inspection data associated with cargo irradiated by a plurality M of successive pulses of X-rays. The inspection data is generated as a result of scanning the cargo using a matrix of detectors. The matrix includes a plurality N of at least two rows of detectors. The successive pulses are generated by a source. The matrix is located at a distance D from the source. The scanning of the cargo includes mutually displacing the cargo and the matrix with a mutual scanning displacement, and detecting, with the matrix, radiation corresponding to the plurality M of successive X-ray pulses irradiating the cargo, during the mutual scanning displacement.

In the inspection data, radiation corresponding to the plurality M of successive pulses irradiating the cargo is arranged in a first order on the plurality N of rows of detectors of the matrix. Successive reconstruction zones for the inspection data are then determined. Each reconstruction zone corresponds to a range of distances from the source in which radiation corresponding to the plurality M of successive pulses irradiating the cargo changes arrangement order. For each determined reconstruction zone, a corresponding intermediate image is generated, and an average image of the intermediate images is also generated. Zones of distortion are then identified in the average image, and, for each zone of distortion neighbourhoods are extracted from each intermediate image, and the neighbourhood having less distortion than the other extracted neighbourhoods is selected.

The selected neighbourhood may be used to correct the average image to generate a final image of the cargo, with reduced distortion.

The selected neighbourhood may be used to determine information relating to the corresponding reconstruction zone. The information may include an ordinal number corresponding to a relative position of the reconstruction zone in the one or more successive reconstruction zones and/or at least one distance from the source corresponding to at least one boundary of the range of distances associated with the reconstruction zone. The information may be used to generate a depth image providing information about a distance of parts of the cargo relative to the matrix.

Embodiments of the disclosure may enable correcting the average image to generate a final image of the cargo, with reduced distortion. Embodiments of the disclosure may enable generating a depth image providing information about a distance of parts of the cargo relative to the matrix and/or the source. Embodiments of the disclosure may enable covering the whole cargo and correct distortion due to the oversampling of some parts of the cargo.

### Details Description of Example Embodiments

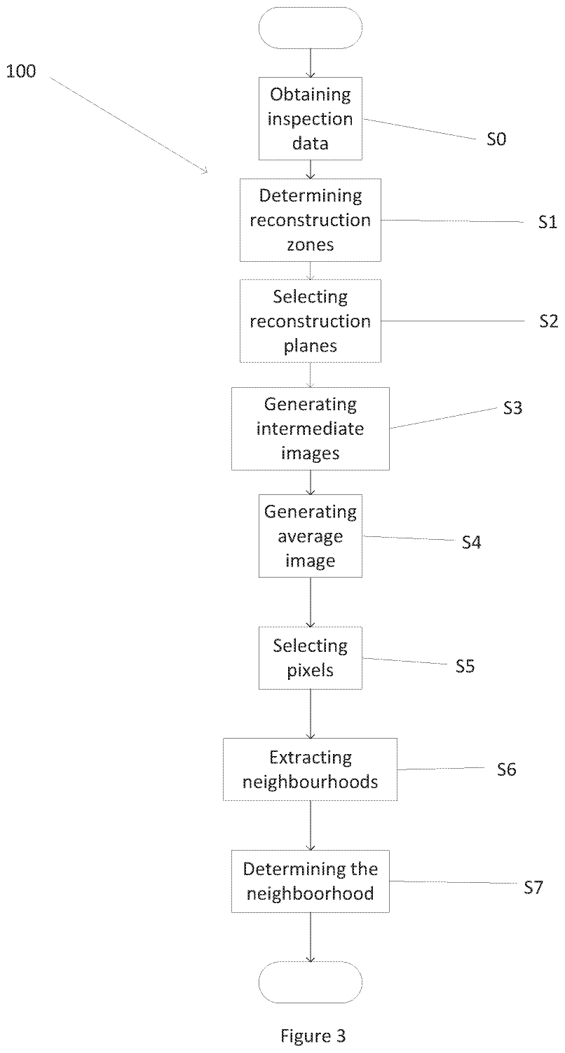

FIG. 3 shows a flow chart illustrating an example method 100 according to the disclosure.

The method 100 is mainly for processing inspection data associated with cargo irradiated by a plurality M of successive pulses of X-rays. The method 100 illustrated in FIG. 3 mainly includes the following steps:

obtaining, at S0, the inspection data;

determining, at S1, one or more successive reconstruction zones for the inspection data;

selecting, at S2, one or more reconstruction planes, based on the determined one or more reconstruction zones;

for each selected reconstruction plane, generating, at S3, an intermediate image of the cargo;

generating, at S4, an average image by averaging all of the generated intermediate images;

on the generated average image, selecting, at S5, one or more pixels having a gradient greater than a predetermined threshold; and

for each one of the pixels selected at S5:

extracting, at S6, a neighbourhood of the selected pixel from each generated intermediate image, and

determining, at S7, in the extracted neighbourhoods, the neighbourhood minimizing a criterion compared with the other extracted neighbourhoods.

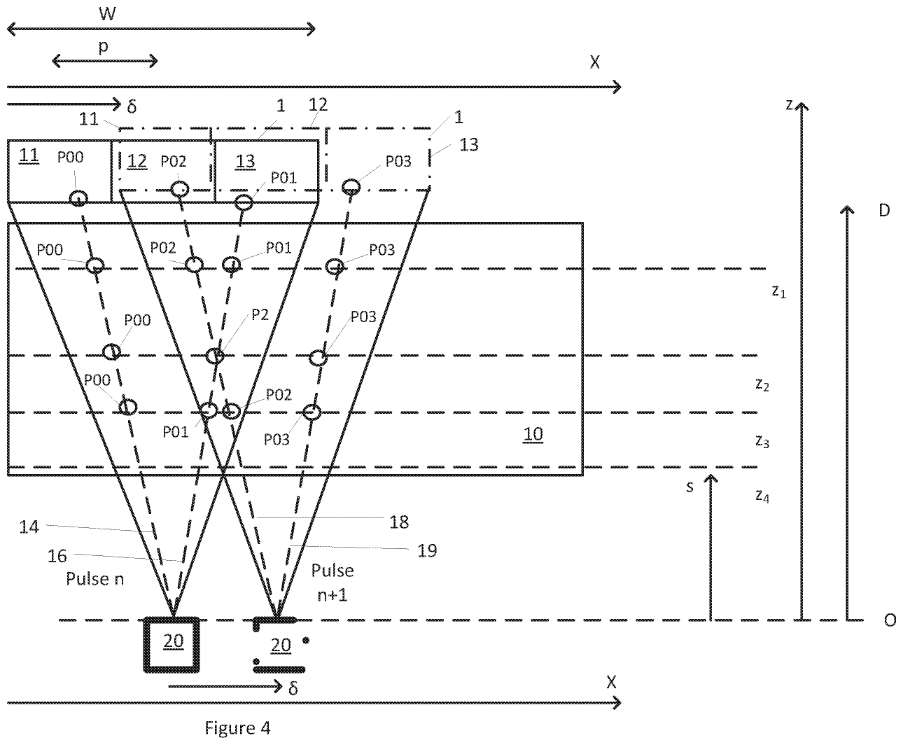

As illustrated in FIG. 4, the inspection data is generated as a result of scanning cargo 10 using a matrix 1 including a plurality N of at least two rows of detectors and a source 20 of the plurality M of successive pulses. In FIG. 4, N=3 and the rows are referenced as 11, 12 and 13. Other numbers N of rows are also envisaged. A pitch p is defined by a distance between a centre of two successive rows or by a width of the matrix 1 divided by N. The matrix 1 is located at a distance D from the source 20.

The pitch p may be a constant in the matrix 1 or may be non-constant in the matrix 1. For example widths of a first row of the matrix 1 and of a last row of the matrix 1 may be larger than width of one or more central rows of the matrix 1. It should be understood that in such a case, the matrix 1 would have several pitch p₁, p₂, . . . , p_{N}. The developments below would still apply.

The scanning of the cargo 10 includes mutually displacing the cargo 10 and the matrix 1 with a mutual scanning displacement. In FIG. 4 shows the matrix 1 and the source 20 moving and the cargo 10 not moving during the mutual scanning displacement. The scanning also includes detecting, with the matrix 1, radiation corresponding to the plurality M of successive X-ray pulses irradiating the cargo 10, during the mutual scanning displacement.

As illustrated in FIG. 4, in the inspection data, radiation corresponding to the plurality M of successive pulses irradiating the cargo is arranged in a first order at a level corresponding to the plurality N of rows 11, 12 and 13 of detectors of the matrix 1, in a direction X corresponding to the mutual scanning displacement.

In FIG. 4, the level corresponding to the plurality N of rows 11, 12 and 13 of detectors corresponds substantially to Z=D.

In FIG. 4, points P00 correspond to radiation direction 14 for pulse n, points P01 correspond to radiation direction 16 for pulse n, points P02 correspond to radiation direction 18 for pulse n+1, and points P03 correspond to radiation direction 19 for pulse n+1.

For example in FIG. 4, a radiation incident on the row 11 corresponding to the n^{th} pulse along direction 14 corresponds to a point P00 and another radiation incident on row 13 along direction 16 corresponds to a point P01. Between the pulse n and the pulse (n+1) the matrix 1 and the source 20 move by a distance δ during the mutual scanning displacement (the cargo 10 is shown as not moving on FIG. 4). The radiation incident on row 11 corresponding to the (n+1)^{th} pulse along direction 18 now corresponds to a point P02 and the radiation incident on row 13 along direction 19 corresponds to a point P03. The directions 14 and 18 correspond to the same direction, just translated between pulses n and n+1. The directions 16 and 19 correspond to the same direction, just translated between pulses n and n+1.

Therefore in the example of FIG. 4, the radiation corresponding to the plurality M of successive pulses irradiating the cargo (here M=2) is arranged in the following first order at the level corresponding to the plurality N of rows 11, 12 and 13 of detectors, in a direction of increasing X: P00, P02, P01 and P03.

In the example of FIG. 4, for covering the whole cargo 10, d must be such that:

$$δ \leq \frac{W . s}{D}$$

with W being a total width of the matrix 1.

Depending on the situation, the displacement δ may vary from one acquisition to another. In other words, δ between two pulses may not be a constant during the mutual scanning displacement. Alternatively or additionally, the displacement δ between two pulses may be a constant during the mutual scanning displacement. For example, in a pass thru mode and at constant source frequency, the displacement δ may depend on the speed of the cargo passing through the scanner. The displacement δ is equal to the instantaneous speed of the cargo divided by the frequency of the source. In a mobile mode, the displacement δ is constant and may be equal to the speed of the scanner divided by the source frequency.

Determining, at S1, the one or more successive reconstruction zones for the inspection data is performed as follows.

The one or more successive reconstruction zones enable selection of one or more reconstruction planes at S2, as explained below.

The one or more reconstruction zones determined at S1 are defined by the experimental conditions (such as the instantaneous speed, the number of matrix rows, the size of the detectors, etc.).

After the one or more reconstruction zones are determined at S1, the one or more reconstruction planes may be selected at S2.

In some examples, one reconstruction plane may be selected for each reconstruction zone. The selected reconstruction plane is located within the determined reconstruction zone. The reconstruction plane may be selected as one of the planes delimiting the reconstruction zone (e.g. the plane closer to the source, but not necessarily). The reconstruction plane may also be selected as being located in the middle of the reconstruction zone, as a non-limiting example.

In some examples, a reconstruction plane may not be selected for each reconstruction zone (e.g. the number of selected reconstruction planes may be lower than the number of determined reconstruction planes), in order to reduce the calculations.

A reconstruction plane enables repositioning acquired data (the data including e.g. the points P01 and P02) at the correct location in the cargo 10. As illustrated in FIG. 4, when acquiring data with the matrix 1, the points e.g. P02 and P01 are in the first order, side by side, along the direction X corresponding to the mutual displacement. Each data corresponds to a detector of the matrix and to a pulse. For example P02 corresponds to detector 11 for pulse (n+1) and P01 corresponds to detector 13 for pulse n. When a plane is chosen for reconstruction of the data, the data are back-projected following a projection lines (e.g. dash lines in FIG. 4) on a plane at a distance z from the source 20, on virtual pixels whose sizes are the detectors' size divided by the corresponding magnification factor z/D. In FIG. 4, point P2, at a distance z=z₂, contributes to the two points P02 and P01 at the level z=D (acquired data). The reconstructions of the points P01 and P02 in the plane z₂ are thus matching in P2. The plane z=z₂ is a correct reconstruction plane. The object P2 corresponding to the data P01 and P02 is located in the plane z=z₂, as the back-projection of P01 and P02 converges in P2 at z=z₂.

On the contrary, if the data P01 and P02 are reconstructed in a plane z=z₁, the two distinct points P01 and P02 are obtained. The plane z=z₁ is thus not a correct plane of reconstruction, because the back-projection of P01 and P02 does not converge. The plane z=D is also not a correct reconstruction plane for P01 and P02.

In some examples, each reconstruction zone corresponds to a range of distances from the source 20 in which radiation corresponding to the plurality M of successive pulses irradiating the cargo 10 is arranged in an order which is different from the first order and different from that of another reconstruction zone of the one or more successive reconstruction zones.

Therefore, the one or more reconstruction zones are zones delimited by planes where the ordering of the data changes.

As already explained, in the simple example of FIG. 4 the first order (at z=D) is P00, P02, P001 and P03. At z=z₁, the order is also P00, P02, P001 and P03. At z=z₂, the order changes to P00, P2 and P03. A first reconstruction zone thus corresponds to distances z such that:

z₂≤z<D.

In the simple example of FIG. 4, at z=z₃, the order is P00, P01, P02 and P03. A second reconstruction zone may thus correspond to distances z such that:

z₄≤Z≤z₂.

As can be seen on FIG. 4, at z=z₄ the order is still P00, P01, P02 and P03, and z₄ is not a plane for which the order is changing. The plane z=z₄ may correspond e.g. to a plane near a face of the cargo 10 located near the source 20. The second reconstruction zone is larger than the range defined by z such that z₄≤z<z₂.

In a more detailed example, a centre of each row (each row being numbered from 0 to N−1) is back-projected in a plane at a distance z (with z being between D and 0), for each pulse.

In a first example, the mutual scanning displacement δ between two pulses is not a constant during the mutual scanning displacement. In such an example, determining the one or more successive reconstruction zones for the inspection data is based on an ordered sequence of radiation position X(k,i,z), along the direction of the mutual scanning displacement. The ordered sequence of radiation position X(k,i,z) for an i^{th} row of detectors and a distance z from the source, for the pulse number k, is such that:

$$X ( k , i , z ) = k . δ ( k ) + i . p . \frac{z}{D} + \frac{p}{2} . ( 1 - \frac{z}{D} )$$

The last term

$$\frac{p}{2} . ( 1 - \frac{z}{D} )$$

is a mere translation shift, and is therefore not a critical parameter, so it may be omitted. The determination of the different reconstruction zones is done by determining the different values of z for which the order of the X(k,i,z) is changing.

In some examples, the method 100 may include, at S1:

determining X(k,i,z) with k varying from 1 to M, iteratively from z being equal to D to a predetermined distance out of a scanning zone, each iteration being performed using an iterative predetermined decrement d; and

for each iteration:

ordering the determined X(k,i,z), and

determining whether the ordering is different from an ordering in a previous iteration, and

if the ordering is different from an ordering in a previous iteration, determining a corresponding distance from the source as a boundary of a reconstruction zone.

The determining may thus include the following steps.

Step 1:

calculation of the X(k,i,z) for z equal to D and with k varying from 1 to the total number M of pulses;

ordering of the X(k,i,D) from the lowest value to the highest value. The ordering is a sequence of (k,i) pairs such as, with M the total number of data equal to a product of the number M of pulses by the number of rows:

0<X(k₁,I₁,D)<X(k₂,I₂,D)<X(k₃,I₃,D)< . . . <X(k_{M},I_{M},D); and

store the ordering.

Step 2:

calculation of the X(k,i,z) for a new z, by decreasing z of a value d such that:

z=D−d; and

ordering.

If the ordering is the same as the ordering stored at step 1, do not change the stored ordering.

If the ordering is not the same as the ordering stored at step 1, D−d is defining a boundary of a new reconstruction zone, store the new ordering and D−d.

Step 3:

calculation of the X(k,i,z) for z, by decreasing z of a value d such that:

z=z−d; and

ordering.

If the order is the same as the ordering at the previous step, do not change the stored ordering.

If the ordering is not the same as the ordering stored at the previous, z−d is defining a boundary of a new reconstruction zone. Store the new ordering and z−d.

Step 4:

iteratively perform step 3 up to a value of z out of the scanning zone (z=D/2 for a mobile scanner, for example).

The above steps do not enable finding exact values of z for which the ordering is changing, but if the value of d is small enough (10 cm, for example) the approximation is good enough.

The sequence of z (z₀, z₁, . . . , zₚ) for which a change in the ordering has been detected is such that:

D>z₀>z₁> . . . >zₚ>zₘᵢₙ.

with zₘᵢₙ a scanning area plane which is closest to the source.

The planes for which the ordering is changing are only known with an accuracy of d and the output of the above steps is a set of depth zones and of ordered sequence of position along the scanning direction. The reconstructions zones are thus as follows:

[zₘᵢₙ,zₚ],[zₚ+d,zₚ₋₁], . . . , [z₁+d,z₀],[z₀+d,D].

In a second example, the reconstructions zones may be thus as follows, as explained in greater detail below:

[zₘᵢⱼₙ,zₚ], . . . ,]z₁,z₀],]z₀,D].

In the second example, the mutual scanning displacement δ between two pulses is a constant during the mutual scanning displacement. In such an example, determining the one or more successive reconstruction zones for the inspection data is based on an ordered sequence of radiation position X(k,i,z), along the direction of the mutual scanning displacement. The ordered sequence of radiation position X(k,i,z) for an i^{th} row of detectors, for the pulse number k, for a plane z, X(k,i,z) is such that:

$$X ( k , i , z ) = k . δ + i . p . \frac{z}{D} .$$

With e.g. N=4 (0≤i≤3), the above positions are a group of four periodic sets of positions, whose common period is:

$$k . δ , k . δ + p . \frac{z}{D} , k . δ + 2. p . \frac{z}{D} , k . δ + 3. p . \frac{z}{D} .$$

In the interval [k·δ,(k+1)·δ[, there are four data, the first data coming from the first row of detector (i=0). The position of the other three rows in the interval may be determined as follows.

The term

$$p . \frac{z}{D}$$

may be such that:

$$p . \frac{z}{D} = m ( z ) . δ + r ( z ) ,$$

with m being integer and r(z) belonging to the interval [0, δ[. Then it follows that:

X(k,i,z)=(k+i·m(z))·δ+i·r(z)

such that m(z)·δ+r(z)=p·(z/D), p being a pitch between the plurality N of at least two rows of detectors.

To determine the order of the rows between [k·δ, (k+1)·δ[, the values of i·r(z) may be ordered. Several cases may be possible. Table 1 below shows a list of the ordering of the positions in the interval [k·δ, (k+1)·δ[ as a function of the values of r(z):

TABLE 1

| | Position 1 | Position 2 | Position 3 | Position 4 |
|---|---|---|---|---|
| 0 ≤ r(z) < δ/3 | row: i = 0 | row: i = 1 | row: i = 2 | row: i = 3 |
| | Pulse number: k | Pulse number: k − m(z) | Pulse number: k − 2 · m(z) | Pulse number: k − 3 · m(z) |
| | Position: k · δ | Position: k · δ + r(z) | Position: k · δ + 2 · r(z) | Position: k · δ + 3 · r(z) |
| δ/3 < r(z) < δ/2 | row: i = 0 | row: i = 3 | row: i = 1 | row: i = 2 |
| | Pulse number: k | Pulse number: k − 3 · m(z) − 1 | Pulse number: k − m(z) | Pulse number: k − 2 · m(z) |
| | Position: k · δ | Position: k · δ + 3 · r(z) − 1 | Position: k · δ + r(z) | Position: k · δ + 2 · r(z) |
| δ/2 < r(z) < 2 · δ/3 | row: i = 0 | row: i = 2 | row: i = 1 | row: i = 3 |
| | Pulse number: k | Pulse number: k − 2 · m(z) − 1 | Pulse number: k − m(z) | Pulse number: k − 3 · m(z) − 1 |
| | Position: k · δ | Position: k · δ + 2 · r(z) − 1 | Position: k · δ + r(z) | Position: k · δ + 3 · r(z) − 1 |
| 2 · δ/3 < r(z) < 1 | row: i = 0 | row: i = 3 | row: i = 2 | row: i = 1 |
| | Pulse number: k | Pulse number: k − 3 · m(z) − 2 | Pulse number: k − 2 · m(z) − 1 | Pulse number: k − m(z) |
| | Position: k · δ | Position: k · δ + 3 · r(z) − 2 | Position: k · δ + 2 · r(z) − 1 | Position: k · δ + r(z) |

For r(z), the interval [0, δ[ may be split in four intervals, in which the positioning is different, as explained below:
between 0 and δ/3, the order is normal with: first row (i=0), second row (i=1), third row (i=2) and fourth row (i=3). Nevertheless, the data corresponding to the second, third and fourth rows are acquired for previous X-ray pulses when m(z)≠0;
between δ/3 and δ/2, the order is different with: first row (i=0), fourth row (i=3), second row (i=1) and third row (i=2). The pulse number for the fourth row has decreased by one;
between δ/2 and 2·δ/3, the order is: first row (i=0), third row (i=2), second row (i=1) and fourth row (i=3); and
between 2·δ/3 and 1, the order is: first row (i=0), fourth row (i=3), third row (i=2) and first row (i=1).

For a range of z such as r(z) is in the above intervals, the reconstruction may use the rows in the same order as above, and a sequence of pixels based on raw data may be the same as above. Therefore for any object of the cargo placed in the range of such z, the reconstruction will be exact. For objects of the cargo placed closer to or further from the source, artefacts or spread out edges appear in the image due to the effect of the reconstruction zones as explained above. The artefacts or the spread out edges are caused by the mis-ordering of the data when the actual z of the reconstructed object in the cargo is not corresponding to the reconstruction plane.

A number of zones generating different orderings may be determined. The number of zones may be calculated first by estimating initial values of m and r for z=D. After the initial values are estimated, values of z (lower than D) for which the ordering between the rows is changing may be calculated.

For an example with N=4 in a mobile mode with an source frequency of 200 Hz, with D being substantially 700 cm and p being 5 mm, δ is 2 mm (for a speed of 40 cm/s), and

p=2·δ+δ/2.

Therefore m(D) is 2 and r(D) is δ/2. In this particular case, the plane of the detectors can be considered as a reconstruction plane because r(D) is corresponding to a boundary of one of the mentioned intervals for r. A next interesting plane may be a plane z₁ for which m(z₁) is still 2 but for which r(z₁) is δ/3:

$$p . \frac{{z}_{1}}{D} = 2. δ + \frac{δ}{3} ⇒ \frac{{z}_{1}}{D} = \frac{7}{3} . \frac{δ}{p}$$

In this particular case (p=5, δ=2), and

$$\frac{{z}_{1}}{D} = \frac{14}{15} ,$$

hence 653.3 cm. By proceeding in the same way with the other boundaries, the following plans may be determined as listed in Table 2.

TABLE 2

| m | r | z | |
|---|---|---|---|
| 2 | δ/3 | 653.3 | cm |
| | 0 | 560 | cm |
| 1 | 2 · δ/3 | 466.6 | cm |
| | δ/2 | 420 | cm |
| | δ/3 | 373.3 | cm |
| | 0 | 280 | cm |
| 0 | 2 · δ/3 | 186.6 | cm |
| | δ/2 | 140 | cm |
| | δ/3 | 93.3 | cm |

In the above example, nine possible plans where the ordering changes are determined. The four plans which are the closest to the source may not be considered, because the cargo may not be that close to the source. Five possible plans where the ordering changes thus remain, and, because the last plane (z=373.3) does not correspond to a face of the cargo closer to the matrix, it means that the depth information may be used to discriminate the cargo in six different reconstruction zones, in a standard mobile mode with four rows with p equal to five millimetres.

The six reconstruction zones are as follows:

zone 1: 653.3<z≤700 (700=D)

zone 2: 560<z≤653.3

zone 3: 466.6<z≤560

zone 4: 420<z≤466.6

zone 5: 373.3<z≤420

zone 6: Zₘᵢₙ<z≤373.3

In a case of pass-thru mode, the number of possible plans may be reduced. In a standard case, with a truck speed of 5 km/h and a source frequency of 200 Hz, the value of δ is around 7 mm, which gives m(D)=0 and r(D)˜0,71. The only possible planes are corresponding to r(z) equal to 2·δ/3, δ/2 and δ/3, which are, in this case, respectively 648.1 cm, 486.1 cm and 324.1 cm. Only the two first one are located in the cargo (324.1 cm is too close to the source) and the depth information will be reduced to two planes for which the order is changing. There are therefore three reconstruction zones as follows:

zone1:z>648.1 cm

zone2:486.1<z≤648.1 cm

zone3:s<z≤486.1 cm

In some examples, for higher frequencies, such as 400 Hz or 600 HZ, at least three planes for which the ordering changes may be determined.

As already stated, the method 100 may include, selecting at S2 one or more reconstruction planes, based on the determined one or more reconstruction zones.

As already stated, the method 100 may also include, for each selected reconstruction plane, generating, at S3, the intermediate image of the cargo.

For a number P of planes where the ordering changes, there may be P+1 reconstruction zones (except when the last plane corresponds to a face of the cargo). If the number of selected planes of reconstruction is equal to the number of zones of reconstruction, a number P+1 of intermediate images may be generated (e.g. reconstructed), the intermediate images corresponding to the P+1 reconstruction planes selected at S2.

The intermediate images may be generated based on the reconstruction plane as follows (in a non-limiting example where it is decided to select one reconstruction plane for each reconstruction zone).

For each of the determined reconstruction zones, a value of z belonging to the corresponding reconstruction zone is selected at S2, such as in the middle or at the boundaries of the reconstruction zone as explained above.

Let z′₀, z′₁, . . . , z′ₚ be a sequence such that:

(z′₁,[z₁+d,z₁₋₁]).

For zone number 1, the ordered sequence of positions is such that:

0<X(k₁₁,I₁₁,z′₁)<X(k₁₁,I₁₂,z′₁)<X(k₁₃,I₁₃,z′₁)< . . . X(k_{1M},I_{1M},z′₁).

M is the total number of data, product of the number of pulses by the number of rows.

At S3, let S(k,I,o) be data acquired par a detector located at a line o of the matrix 1 and the row I during the pulse k, the line o of the reconstructed image for the zone number 1 will be the sequence:

S(k₁₁,I₁₁,o),S(k₁₂,I₁₂,o),S(k₁₃,I₁₃,o), . . . ,S(k_{1M},I_{1M},o).

In the reconstruction, the sequence of data is not depending on the choice of z′₁. However X(k_{1M},I_{1M},z′₁) may be calculated if required.

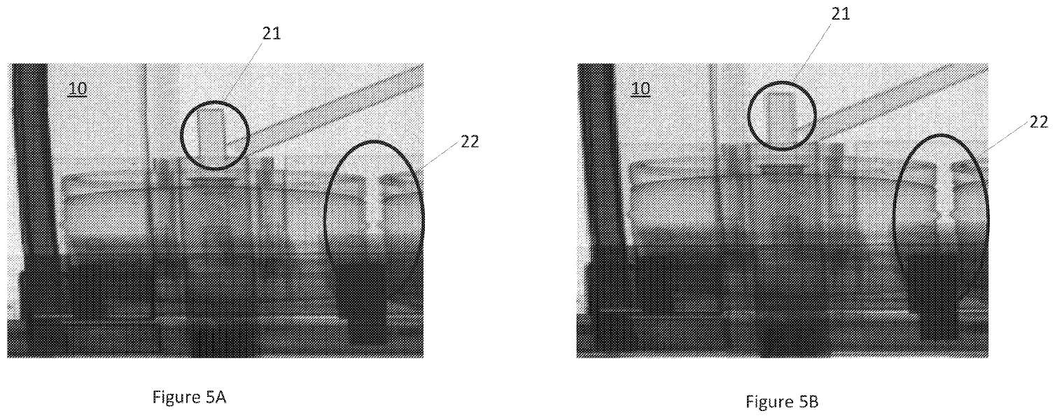

As illustrated in FIG. 5A, reconstruction of an object in an area 21 located at a distance z=3.9 m from the source, using a reconstruction plane located at 3.9 m from the source, generates an intermediate image without an improper gradient in a direction corresponding to the mutual scanning displacement. As illustrated in FIG. 5A, reconstruction of an object in an area 22 located at a distance z=6.5 m from the source, using the reconstruction plane located at z=3.9 m from the source, generates the intermediate image with a gradient (e.g. artefacts or spread out edges) in a direction corresponding to the mutual scanning displacement.

As illustrated in FIG. 5B, reconstruction of the object in the area 21 located at a distance z=3.9 m from the source, using a reconstruction plane located at 6.5 m from the source, generates an intermediate image with a gradient (e.g. artefacts or spread out edges) in a direction corresponding to the mutual scanning displacement. As illustrated in FIG. 5B, reconstruction of the object in the area 22 located at a distance z=6.5 m from the source, using the reconstruction plane located at 6.5 m from the source, generates the intermediate image without an improper gradient in a direction corresponding to the mutual scanning displacement.

Therefore as illustrated in FIGS. 5A and 5B, when the reconstruction zone does not correspond to the object location in the cargo, objects with relatively high horizontal gradient show artefacts or have spread out edges in the image.

As already stated, the method 100 may include generating, at S4, an average image by averaging all of the generated intermediate images.

On the generated average image, the selecting, at S5, of the one or more pixels having a horizontal gradient (i.e. in the direction corresponding to the mutual scanning displacement) which is greater in absolute value than a predetermined threshold enable identification of vertical or oblique edges.

The absolute value of the horizontal gradient can be calculated for example by a standard formula of horizontal variation along direction x of pixel (i,j) such as:

|∇_{x,i,j}|=|I_{i+1,j}+I_{i-1,j}|/2

I_{i,j} being the intensity of the pixel (i,j) with i being the horizontal coordinate (i.e. in the scanning direction).

Alternatively or additionally, the absolute value of the horizontal grandient can also be calculated based on a Sobel mask.

The threshold Th can be equal to a parameter A having constant value. To take into account the noise dependency of the signal, the threshold Th can also be implemented as a function of the pixel average in a neighbourhood such that:

$${Th}_{i , j} = A . f ( {\sum }_{neighbourhood} {I}_{{i}^{′} , {j}^{′}} )$$

In such a case the threshold Th varies from one pixel to another.

The predetermined parameter A may be chosen by a user of an inspection system implementing the method according to any aspects of the disclosure.

As already stated, the method 100 may include, for each one of the pixels selected at S5, extracting, at S6, a neighbourhood of the selected pixel from each generated intermediate image. For each of the pixels selected at S5, S6 enables to extract P+1 neighbourhoods (e.g. thumbnails). In some examples the neighbourhood may correspond to an 8-connected neighbourhood.

As already stated, the method 100 may include, determining, at S7, in the extracted neighbourhoods, the neighbourhood minimizing a criterion compared with the other extracted neighbourhoods. The minimal criterion may be associated for examples with sharper edges (e.g. fewer artefacts) in the determined neighbourhood. The determined neighbourhood may thus have fewer artefacts than the other extracted neighbourhoods.

As already discussed with reference to FIGS. 5A and 5B, horizontal gradients (e.g. corresponding to artefacts) appear in vertical edges of the objects and look like discontinuous lines when the objects are reconstructed with a reconstruction plane which is different from the reconstruction plane to which the objects belong. Based on this observation, the criterion to minimize may be an energy E which may be determined as follows. The energy E includes two terms: a first term being a horizontal gradient, squared, and a second term being a penalization function as defined as followed. The neighbourhood having the lowest energy E is selected.

In some embodiments, determining at S7 the neighbourhood having the minimal criterion may include:

for each value I_{ij,p} of the p^{th} generated intermediate image Ip, in coordinates (i,j) corresponding to one of the selected pixels, and for all of the generated intermediate image Ip, determining the energy E such that:

E(I_{ij,p})=(∇ₓI)²_{ij,p}+g(I_{ij,p})

with the first term being a gradient along the direction x of the mutual scanning displacement, squared, and

a second term being a penalization function g configured to penalize, in the minimizing, neighbourhoods with different gray levels.

In some examples, the determining at S7 may also include selecting the neighbourhood associated with the generated intermediate image Ip having a minimal determined energy.

In some examples, the penalization function g is a Geman-McClure function defined by:

$$g ( {I}_{ij , p} ) = {\sum }_{k ∈ {N}_{ij}^{6}} ρ ( {r}_{k} , σ ( {r}_{k} ) )$$

with k is a value used to index the pixels of the neighbourhood of the pixel (i,j). In the Nᵢⱼ⁶ neighbourhood, they correspond to the pixels (i−1, j+1),(i−1,j),(i−1,j−1),(i+1,j+1),(i+1,j),(i+1,j−1), k is varying from 1 to 6,

with

$$ρ ( r , σ ) = \frac{{r}^{2}}{{r}^{2} + {σ}^{2}} ,$$

rₖ={Iₖ−Iᵢⱼ|k∈Nᵢⱼ⁶}, Iₖ being values of the images in the neighbourhood Nᵢⱼ⁶, Iᵢⱼ being the value of the image for the current pixel (i,j),

and

σ(rₖ) is the standard deviation of the value of rₖ on the neighbourhood

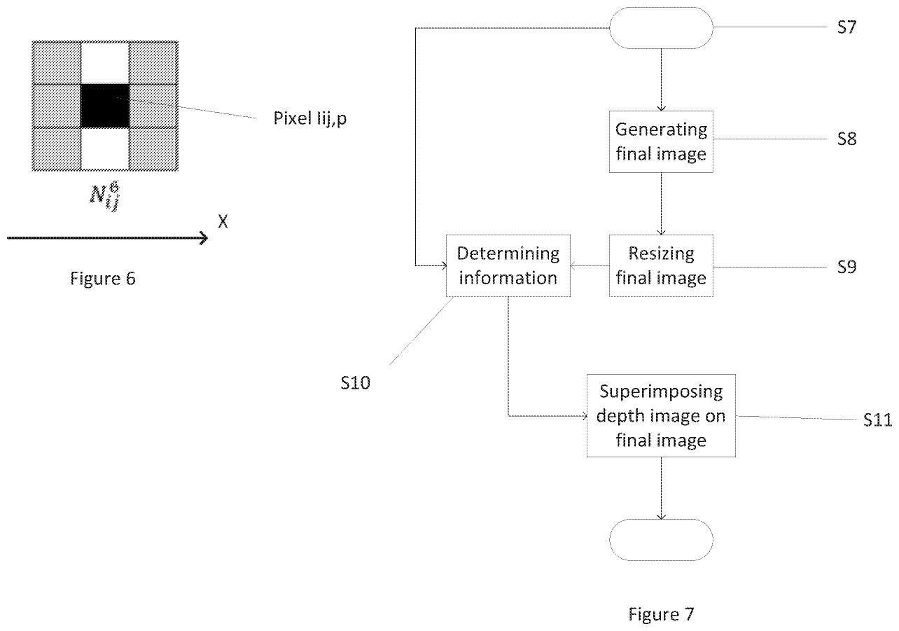

As illustrated in FIG. 6, N⁶ᵢⱼ a neighbourhood having white pixels above and below the pixel of value I_{ij,p} in a direction perpendicular to the direction of the mutual scanning displacement x, and having grey pixels elsewhere in the neighbourhood. Nᵢⱼ⁶ is the neighborhood of the pixel located in (i,j), indexed by k from 1 to 6, and whose constitutive pixels are the three closest pixels to (i,j) located in the column before and the three closest pixels to (i,j) located in the column after.

The function g penalizes neighbourhoods with different gray levels. If gray lines in FIG. 6 are discontinuous lines due to stereoscopic effect, rₖ will have high values and g will also have high values, which will penalize E. E will not have high values if the neighbourhood is a homogeneous area.

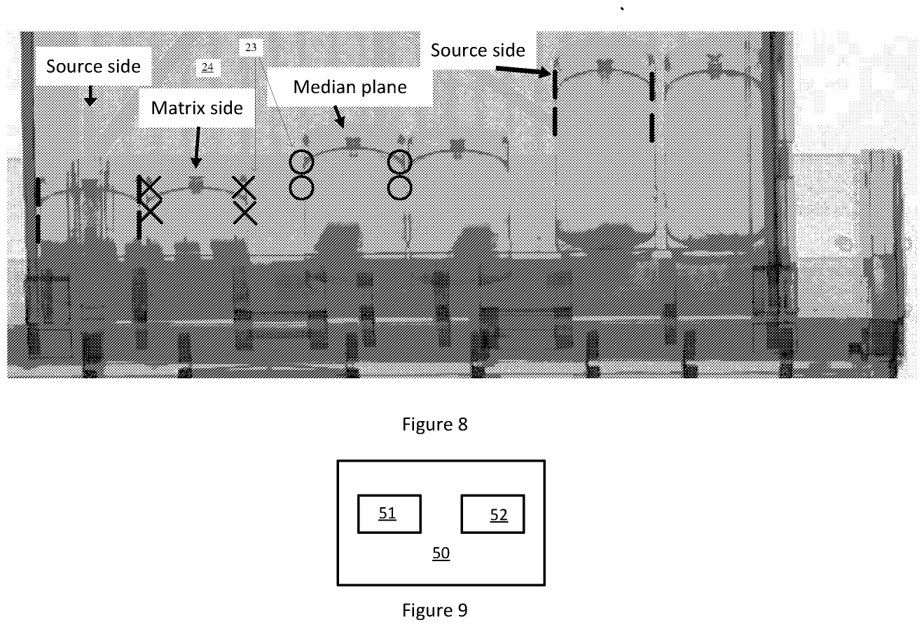

In a first example, as illustrated in FIG. 7, the method 100 may further include generating, at S8, a final image of the cargo. The final image may correspond to a corrected average image. An example final image 24 is illustrated in FIG. 8.

In some examples, generating at S8 the final image including pixels may include:

assigning, for each pixel of the final image corresponding to one of the selected pixels, a value of the pixel in the generated intermediate image corresponding to the neighbourhood determined at S7; and

assigning, for each other pixel of the final image, a value of the pixel in the average image determined at S4 or in the generated intermediate image corresponding to a reconstruction zone closest to the matrix determined at S3.

The method 100 may include resizing, at S9, the generated final image, in the direction corresponding to the mutual scanning displacement and/or perpendicularly to the direction corresponding to the mutual scanning displacement, to obtain a width on height ratio corresponding to a median plane of the cargo, the median plane being substantially parallel to the matrix.

Alternatively or additionally, in a second example, as illustrated in FIG. 7, the method 100 may further include, for each pixel corresponding to one of the pixels selected at S5, determining, at S10, information relating to the determined reconstruction zone corresponding to the determined neighbourhood having the fewer artefacts.

As illustrated in FIG. 7, the determining at S10 may be performed after the step S7. In other words, the pixel corresponding to one of the selected pixels may include at least one pixel of the generated average image. Alternatively or additionally, the determining at S10 may be performed after the step S8 or the step S9. In other words, the pixel corresponding to one of the selected pixels may include at least one pixel of the final image of the cargo.

In some examples, the determined information relating to the determined reconstruction zone includes:

an ordinal number corresponding to a relative position of the determined reconstruction zone in the one or more successive reconstruction zones and/or a depth zone, each pixel not corresponding to one of the selected pixels not being associated with an ordinal number and/or a depth zone; and/or

at least one distance from the source corresponding to at least one boundary of the range of distances associated with the determined reconstruction zone in the one or more successive reconstruction zones.

In some examples, determining at S10 the information may further include:

predetermining a number of possible depth zones, the number of possible depth zones being equal to or smaller than a number of the determined one or more successive reconstruction zones;

merging the determined one or more successive reconstruction zones into one or more depths zones, a number of the one or more depths zones corresponding to the number of possible depth zones;

assigning a merged depth zone to each pixel corresponding to one of the selected pixels, based on the determined information; and

generating a depth image based on the assigning, each pixel not corresponding to one of the selected pixels not being assigned to a merged depth zone.

In some examples the number of possible depth zones may be chosen by the user of the inspection system implementing the method according to any aspects of the disclosure, and may be equal to e.g. three (corresponding to e.g. a detector side, a middle of the cargo or median plane, and/or an source side).

In the merging, for example, if we have the following reconstruction zones:

[zₘᵢⱼₙ,zₚ],[zₚ+d,zₚ₋₁], . . . ,[z₁+d,z₀],[z₀+d,D]

and if the predetermined number of possible depth zones is three, the merged depth zones may be as follows:

[zₘᵢⱼₙ,zₚ₁],[zₚ₁+d,zₚ₂],[zₚ₂+d,D]

with z₁₁ and z₁₂ depth values from the sequence z₀, z₁, . . . , zₚ.

In the example of FIG. 7, the method 100 may further include superimposing, at S11, the depth image generated at S10 on the final image of the cargo generated at S8 and/or S9.

FIG. 8 illustrates a depth image 23 superimposed on the final image 11 of the cargo.

In the example of FIG. 8, the predetermined number of possible depth zones is three, and the merged depth zones correspond to “source side”, “median plane” and “matrix side”. The depth image 23 may include visual markers to indicate the depth information. In the example of FIG. 8, the depth image 23 may include straight lines to indicate that the corresponding object is located on the source side in the cargo, circles to indicate that the corresponding object is located on the median plane in the cargo, and crosses to indicate that the corresponding object is located on the source side in the cargo. Other visual markers may be envisaged, such as colours (e.g. red for source side, green for median plane and blue for matrix side, as non-limiting examples).

The generated depth image may be noisy, because e.g. of spurious variations of depth from one pixel to another pixel in the neighbourhoods. The method may thus further include denoising the generated depth image by forcing adjacent pixels to belong to a common depth zone. In some examples, the forcing may be based on the following constraint: two pixels vertically adjacent cannot belong to two different reconstruction planes.

As illustrated in FIG. 9, the disclosure also relates to a system 50 including a processor 51 and a memory 52 storing instructions which, when executed by the processor, enable the processor to perform a method according to any aspect of the present disclosure.

In some examples the source is configured to irradiate the cargo with at least two different levels of energy for material discrimination. In such examples, the method of any aspect of the disclosure may be performed for each of the at least two different levels of energy.

In the examples above, the inspection radiation source may include an X-ray generator. The energy of the X-rays may be between 100 keV and 15 MeV, and the dose rate may be between 2 mGy and 20 Gy (Gray) per minute at one meter from the source. The maximum X-ray energy of the X-ray source may be e.g., between 100 keV and 9.0 MeV, typically e.g., 2 MeV, 3.5 MeV, 4 MeV, or 6 MeV, for a steel penetration capacity e.g., between 40 mm to 400 mm, typically e.g., 300 mm (12 in). The dose may be e.g., between 20 mGy and 120 mGy. In other examples, the maximum x-ray energy of the X-ray source may be e.g., between 4 MeV and 10 MeV, typically e.g., 9 MeV, for a steel penetration capacity e.g., between 300 mm to 450 mm, typically e.g., 410 mm (16.1 in). In some examples, the dose may be 17 Gy.

An inspection system implementing the method may further include other types of detectors, such as optional gamma and/or neutrons detectors, e.g., adapted to detect the presence of radioactive gamma and/or neutrons emitting materials within the cargo 10, e.g., simultaneously to the X-ray inspection.

1. A method for processing inspection data associated with cargo irradiated by a plurality M of successive pulses of X-rays, comprising:
obtaining the inspection data, the inspection data being generated as a result of scanning the cargo using a matrix comprising a plurality N of at least two rows of detectors, and a source of the plurality M of successive pulses, the matrix being located at a distance D from the source, the scanning comprising:
mutually displacing the cargo and the matrix with a mutual scanning displacement, and
detecting, with the matrix, radiation corresponding to the plurality M of successive X-ray pulses irradiating the cargo, during the mutual scanning displacement, wherein, in the inspection data, radiation corresponding to the plurality M of successive pulses irradiating the cargo and detected by the plurality N of at least two rows of detectors is arranged in a first order corresponding to a level of the matrix, in a direction corresponding to the mutual scanning displacement;
determining one or more successive reconstruction zones for the inspection data, wherein each reconstruction zone corresponds to a range of distances from the source in which radiation corresponding to the plurality M of successive pulses irradiating the cargo is arranged in an order which is different from the first order and different from that of another reconstruction zone of the one or more successive reconstruction zones;
selecting one or more reconstruction planes, based on the determined one or more reconstruction zones;
for each selected reconstruction plane, generating an intermediate image of the cargo;
generating an average image by averaging all of the generated intermediate images;
on the generated average image, selecting one or more pixels having a gradient, in a direction corresponding to the mutual scanning displacement, greater in absolute value than a predetermined threshold; and
for each one of the selected pixels:
extracting a neighbourhood of the selected pixel from each generated intermediate image, and
determining, in the extracted neighbourhoods, the neighbourhood minimizing a criterion compared with the other extracted neighbourhoods.

2. The method of claim 1, further comprising generating a final image of the cargo corresponding to a corrected average image, the final image comprising pixels and generating the final image comprising:
assigning, for each pixel of the final image corresponding to one of the selected pixels, a value of the pixel in the generated intermediate image corresponding to the determined neighbourhood; and
assigning, for each other pixel of the final image, a value of the pixel in the average image or in the generated intermediate image corresponding to a reconstruction zone closest to the matrix.

3. The method of claim 1, further comprising resizing the generated final image, in the direction corresponding to the mutual scanning displacement and/or perpendicularly to the direction corresponding to the mutual scanning displacement, to obtain a width on height ratio corresponding to a median plane of the cargo, the median plane being substantially parallel to the matrix.

4. The method of claim 1, further comprising, for each pixel corresponding to one of the selected pixels:
determining information relating to the determined reconstruction zone corresponding to the determined neighbourhood.

5. The method of claim 4, wherein determining the information further comprises:
predetermining a number of possible depth zones, the number of possible depth zones being equal to or smaller than a number of the determined one or more successive reconstruction zones;
merging the determined one or more successive reconstruction zones into one or more depths zones, a number of the one or more depths zones corresponding to the number of possible depth zones;
assigning a merged depth zone to each pixel corresponding to one of the selected pixels, based on the determined information; and
generating a depth image based on the assigning, each pixel not corresponding to one of the selected pixels not being assigned to a merged depth zone.

6. (canceled)

7. The method of claim 5, further comprising:
denoising the generated depth image by forcing adjacent pixels to belong to a common depth zone.

8. The method of claim 4, wherein the pixel corresponding to one of the selected pixels comprises:
at least one pixel of the generated average image, and/or
at least one pixel of the final image of the cargo.

9. The method of claim 4, wherein the determined information relating to the determined reconstruction zone comprises:
an ordinal number corresponding to a relative position of the determined reconstruction zone in the one or more successive reconstruction zones and/or a depth zone, each pixel not corresponding to one of the selected pixels not being associated with an ordinal number and/or a depth zone; and/or
at least one distance from the source corresponding to at least one boundary of the range of distances associated with the determined reconstruction zone in the one or more successive reconstruction zones.

10. The method of claim 1, wherein a mutual scanning displacement δ between two pulses is a constant during the mutual scanning displacement, and wherein determining the one or more successive reconstruction zones for the inspection data is based on an ordered sequence of radiation position X(k,i,z), along a direction of the mutual scanning displacement, associated with an i^{th} row of detectors and a distance z from the source, for the pulse number k, such that:
X(k,i,z)=(k+i·m(z))·δ+i·r(z)
with m being an integer,
r(z) belonging to the interval [0, δ[, and
such that m(z)·δ+r(z)=p·(z/D), p being a pitch between the plurality N of at least two rows of detectors.

11. The method of claim 1, wherein a mutual scanning displacement δ between two pulses is not a constant during the mutual scanning displacement, and wherein determining the one or more successive reconstruction zones for the inspection data is based on an ordered sequence of radiation position X(k,i,z), along a direction of the mutual scanning displacement, corresponding to an i^{th} row of detectors and a distance z from the source, for the pulse number k, such that:
$$X ( k , i , z ) = k . δ ( k ) + i . p . \frac{z}{D} + \frac{p}{2} . ( 1 - \frac{z}{D} )$$
with p being a pitch between the plurality N of at least two rows of detectors.

12. The method of claim 11, further comprising:
determining X(k,i,z) with k varying from 1 to M, iteratively from z being equal to D to a predetermined distance out of a scanning zone, each iteration being performed using an iterative predetermined decrement d; and
for each iteration:
ordering the determined X(k,i,z),
determining whether the ordering is different from an ordering in a previous iteration, and
if the ordering is different from an ordering in a previous iteration, determining a corresponding distance from the matrix as a boundary of a reconstruction zone.

13. The method of claim 1, wherein determining the neighbourhood having the minimal criterion comprises:
for each value I_{ij,p} of the p^{th} generated intermediate image Ip, in coordinates (i,j) corresponding to one of the selected pixels, and for all of the generated intermediate image Ip, determining energy E such that:
E(I_{ij,p})=(∇ₓI)²_{ij,p}+g(I_{ij,p})
with a first term being a gradient along the direction x of the mutual scanning displacement, squared, and
a second term being a penalization function g configured to penalize, in the minimizing, neighbourhoods with different gray levels; and
selecting the neighbourhood associated with the generated intermediate image Ip having a minimal determined energy.

14. The method of claim 13, wherein g is a Geman-McClure function defined by:
$$g ( {I}_{ij , p} ) = {\sum }_{k ∈ {N}_{ij}^{6}} ρ ( {r}_{k} , σ ( {r}_{k} ) )$$
with Nᵢⱼ⁶ being the neighborhood of the pixel located in (i,j), indexed by k from 1 to 6, and whose constitutive pixels are the three closest pixels to (i,j) located in the column before and the three closest pixels to (i,j) located in the column after,
$$ρ ( r , σ ) = \frac{{r}^{2}}{{r}^{2} + {σ}^{2}} ,$$
rₖ={Iₖ−Iᵢⱼ|k∈Nᵢⱼ⁶}, Iᵢⱼ being the image value at pixel (i,j) and Iₖ the image values for the pixel in the neighbourhood Nᵢⱼ⁶, and
σ(rₖ) is the standard deviation of the value of rₖ on the neighbourhood.

15. The method of claim 1, wherein the neighbourhood is an 8-connected neighbourhood.

16. The method of claim 1, wherein the source is configured to irradiate the cargo with at least two different levels of energy for material discrimination, the method for processing the inspection data being performed for each of the at least two different levels of energy.

17. The method of claim 1, wherein
the absolute value of the gradient along direction x is such that:

|∇_{x,i,j}|=|I_{i+1,j}−I_{i-1,j}|/2
with I_{i,j} being an intensity of a pixel (i,j), with i being the coordinate in the direction x corresponding to the mutual scanning displacement, and/or
the absolute value of the gradient is calculated based on a Sobel mask.

18. The method of claim 17, any of the previous claims, wherein the predetermined threshold is a constant value or varies from one pixel to another pixel.

19. A system comprising:
a processor; and
a memory storing instructions which, when executed by the processor, enable the processor to perform a method for processing inspection data associated with cargo irradiated by a plurality M of successive pulses of X-rays, comprising:
obtaining the inspection data, the inspection data being generated as a result of scanning the cargo using a matrix comprising a plurality N of at least two rows of detectors, and a source of the plurality M of successive pulses, the matrix being located at a distance D from the source, the scanning comprising:
mutually displacing the cargo and the matrix with a mutual scanning displacement, and detecting, with the matrix, radiation corresponding to the plurality M of successive X-ray pulses irradiating the cargo, during the mutual scanning displacement, wherein, in the inspection data, radiation corresponding to the plurality M of successive pulses irradiating the cargo and detected by the plurality N of at least two rows of detectors is arranged in a first order corresponding to a level of the matrix, in a direction corresponding to the mutual scanning displacement;
determining one or more successive reconstruction zones for the inspection data, wherein each reconstruction zone corresponds to a range of distances from the source in which radiation corresponding to the plurality M of successive pulses irradiating the cargo is arranged in an order which is different from the first order and different from that of another reconstruction zone of the one or more successive reconstruction zones;
selecting one or more reconstruction planes, based on the determined one or more reconstruction zones;
for each selected reconstruction plane, generating an intermediate image of the cargo;
generating an average image by averaging all of the generated intermediate images;
on the generated average image, selecting one or more pixels having a gradient, in a direction corresponding to the mutual scanning displacement, greater in absolute value than a predetermined threshold; and
for each one of the selected pixels:
extracting a neighbourhood of the selected pixel from each generated intermediate image, and
determining, in the extracted neighbourhoods, the neighbourhood minimizing a criterion compared with the other extracted neighbourhoods.

20. (canceled)

21. (canceled)
